(19) United States
(12) Patent Application Publication
(10) Pub. No.: US 2009/0238786 A1
(43) Pub. Date: Sep. 24, 2009

(54) COSMETIC FORMULATIONS INCLUDING SOLID ANTIPERSPIRANTS

(76) Inventors: Ian J. FISHMAN, Los Angeles, CA (US); Eric A. Fishman, Los Angeles, CA (US)

Correspondence Address: KONRAD RAYNES & VICTOR, LLP, 315 S. BEVERLY DRIVE, # 210, BEVERLY HILLS, CA, 90212, US

(21) Appl. No.: 12/052,793

(22) Filed: Mar. 21, 2008

(51) Int. Cl. A61K 8/30; A61Q 15/00

(52) U.S. Cl. 424/65

(57) ABSTRACT

Cosmetic compositions such as solid antiperspirants and method for their manufacture are described. One such composition includes 1-25 weight percent of an antiperspirant drug active and 10-40 weight percent water. The composition also includes 25-60 weight percent of a humectant and 5-20 weight percent of a fatty acid. The composition also includes 2-10 weight percent of an alkoxylated alcohol and 0.1-10 weight percent of a neutralizer. Other embodiments are described and claimed.

### RELATED ART

Antiperspirant systems have commonly been comprised of a combination of ingredients including an antiperspirant drug active to control perspiration, together with other ingredients such as waxes and/or gelling agents, and emollients. Antiperspirants have been manufactured in numerous forms, including solids and gels. Typical active drug ingredients include aluminum, zirconium, and aluminum zirconium salts. Typical waxes and gelling agents for forming solid and gel form antiperspirants include fatty alcohols, hydrocarbon waxes, and synthetic materials such as silicones.

Many antiperspirant products such as sticks and the like, are dry, tacky, and are uncomfortable to use. Often, the product leaves a white residue on the skin and clothes of the user. They do not glide well under the arms, and tend to clump up during application. Others, such as certain gel products, are soft and sticky. Such antiperspirant products often leave the consumer unsatisfied.

### DETAILED DESCRIPTION OF PREFERRED EMBODIMENTS

Certain embodiments of the present invention relate to and include compositions and methods for forming antiperspirant products. One example of such a system is solid antiperspirants. Solid antiperspirants include a stick or firm bar of material that retains its integrity while it is being applied to the body. Upon application to the skin, a thin layer of the solid material is transferred onto the skin surface.

Certain embodiments relate to solid antiperspirant compositions including an antiperspirant such as a metal salt, together with effective amounts of a humectant, water, a fatty acid, an alkoxylated alcohol, and a pH adjuster (for example, an inorgainc base).

The antiperspirant drug active ingredient used in various embodiments may include FDA approved antiperspirant drug actives, including, for example, various aluminum, zirconium, and aluminum zirconium salts. Aluminum chlorohydrate (INCI name) is an example of one such antiperspirant salt. Other examples include, but are not limited to, aluminum chloride, aluminum chlorohydrex polyethylene glycol, aluminum chlorohydrex propylene glycol, aluminum dichlorohydrex polyethylene glycol, and aluminum sesquichlorohydrex polyethylene glycol (INCI names). The antiperspirant drug active may be present in an amount of up to about 25 weight percent.

Water is used as a solvent in various embodiments. Generally, the quantity of water may depend on the specific fatty acid and neutralizers used, as well as any other additives included in the formulation. The water may be present in certain embodiments in a range of about 2 to about 60 weight percent, with certain preferred embodiments including about 10 to about 40 weight percent water. Deionized water is preferred.

The water also acts to provide a product with improved feel when applied to the body. Certain preferred embodiments use at least 10 weight percent water because lower quantities may yield a product that feels undesirably dry on the skin. In addition, when lower quantities of water are used, it may be more difficult to dissolve the drug active.

Many conventional stick antiperspirants do not include water, because when the drug active ingredient is mixed with water, an acidic solution is formed. This acidity inhibits the formulation from readily solidifying, thus making stick formation difficult.

Various embodiments also include a pH adjuster or neutralizer component, which enables the composition to readily form a solid. One example of such a component is sodium hydroxide (INCI name), which acts to neutralize the acid in the formulation of certain embodiments. A variety of other materials, both organic and inorganic, may be used as a pH adjuster or neutralizer, including, but are not limited to, ammonium hydroxide, triethaneolamine (TEA), and potassium hydroxide (INCI names). Certain embodiments utilize the pH adjuster or neutralizer in an amount of about 0.05 to 25%, with certain preferred embodiments including about 0.1 to about 10 weight percent.

Various embodiments also include a humectant component. Humectants are conditioning materials for rehydrating the skin and aiding in retention of water by the skin. Preferred embodiments may include a humectant component comprising propylene glycol (INCI name). Other humectant materials may also be used, including, but not limited to glycerine, sorbitol, and butylene glycol (INCI names). In certain embodiments the humectant ingredient may also act as a solvent for the active antiperspirant ingredient. Certain embodiments include the humectant being present in a range of about 10 to about 70 weight percent. Certain preferred embodiments include the humectant present in an amount of about 25-60 weight percent.

Various embodiments also include a fatty acid component, such as sodium stearate (INCI name). This component is believed to act as an emulsifying agent, which can attract both water and oil at the same time, thus promoting emulsification. Other fatty acids may be used, including, but not limited to, Ceteareth-2 through Ceteareth-22, Ceteth-1 through Ceteth 7, Ceteth-10, and Ceteth 12-18 (INCI names). The fatty acid component may in certain embodiment be present in an amount of about 1 to about 50 weight percent, with certain more preferred embodiments including about 5 to about 20 weight percent.

Various embodiments also utilize an alkoxylated alcohol component such as Steareth-20 (INCI name), also known as polyoxyethylene (20) stearyl ether. Steareth-20 may also be characterized as a fatty alcohol ethoxylate. The Steareth-20 also is believed to act as an emulsifying agent. Other alkoxylated alcohols may be used, including, but not limited to, Steareth-21, Steareth-25, Steareth-27, Steareth-30, Steareth-40, Steareth-50, and Steareth-80 (INCI names). The alkoxylated alcohol may in certain embodiments be present in an amount of about 1 to about 40 weight percent, with more preferred embodiments including about 2 to about 10 weight percent.

In certain preferred embodiments, multiple emulsifier ingredients are used, for example, both a fatty acid and an alkoxylated alcohol.

As described above, water may be used in certain embodiments as a solvent. Other examples of solvents that could be used with water include, but are not limited to, alcohol (preferably denatured), mineral oil, cyclopentasiloxane, and propylene glycol.

Various embodiments may also include additives to impart properties such as a desired smell, etc., to the product. For example, certain embodiments use a fragrance, in oil form, which may be present in varying amounts depending on the specific fragrance used and the desired strength. Certain embodiments may use, for example, up to about 10 weight percent fragrance. A variety of additional ingredients may be optionally included in the product, in order to impart desirable characteristics. For example, embodiments may include additives such as, for example, additional humectants, moisturizers, proteins, vitamins, herbal extracts, colorants, antibacterials, and the like, as known in the art.

Cosmetic stick formulations such as solid antiperspirants in accordance with embodiments described herein may be formed to be translucent or opaque, depending at least in part on the quantities of certain ingredients included. For example, varying the amount of the antiperspirant drug active ingredient may affect the translucency of the antiperspirant stick. In certain embodiments, as the level of antiperspirant drug active is increased, the level of translucency is decreased. In another example, different additives, for example, fragrances, may also affect the translucency of the antiperspirant stick.

One example of an embodiment of an antiperspirant stick formulation is set forth in Table 1 below. The formulation set forth in Table 1 may be utilized to form an antiperspirant stick having good feel on the skin.

TABLE 1
Antiperspirant Stick Formulation.

| Ingredient | (wt %) |
|---|---|
| Hydrous Aluminum | 6.500 (5.2% aluminum |
| Chlorohydrate | chlorohydrate, anhydrous) |
| Propylene Glycol | 52.750 |
| Water (Aqua Purificata) | 20.875 |
| Purified | |
| Sodium Stearate | 12.000 |
| Steareth-20 | 5.000 |
| Sodium Hydroxide | 0.875 |
| Fragrance (Parfum) | 2.000 |
| total | 100.000 |

Certain embodiments also relate to methods for forming cosmetic products, for example, an antiperspirant having the ingredients listed in Table 1. In one method embodiment for the preparation of an antiperspirant stick, in a main mixing tank, a first component including the antiperspirant drug active is prepared. This is accomplished by combining in a main mixing tank 6.5 pounds of aluminum chlorohydrate (available as hydrous aluminum chlorohydrate under the trade name MACROSPHERICAL 95 ALUMINUM CHLOROHYDRATE, from Reheis, Inc.), with 22.75 pounds of propylene glycol (available from Dow Chemical Co.), and 20 pounds of water. The components are heated to a temperature of 75-80° C. and mixed for 10-15 minutes or until all solids are dissolved.

Next, the pH is adjusted by adding 1.75 pounds of sodium hydroxide (available as SODIUM HYDROXIDE 50%, from Ashland Distribution Co. & Ashland Specialty Chemical Co.) to the batch in the main mixing tank at 75-80° C. and mixing for 10-15 minutes or until the batch is uniform. In a separate container, 30 pounds of propylene glycol (available from Dow Chemical Co.), 12 pounds of sodium stearate (available as SODIUM STEARATE C-7, from Crompton Corp.), and 5 pounds of Steareth-20 (available under the trade name VOLPO S-20, from Croda, Inc.), are mixed and heated to 75-80° C. The contents of the separate container are then combined with the batch in the main mixing tank, at 75-80° C., and mixed to 10-15 minutes or until uniform. The batch is then cooled to 60-65° C. If desired, a fragrance may then be added while the batch is at 60-65° C. In certain embodiments, 2 pounds of fragrance is added to the batch and mixed for 5-10 minutes or until the batch is uniform. The batch, in this embodiment, may have a pH in the range of 8.5-9.5. The batch may then be poured into individual containers. To speed up the solidification of the product, the product may be put through a chill tunnel at approximately 10-15° C., and then brought to room temperature. Any suitable container for a solid antiperspirant product may be used, for example, a conventional stick or barrel dispenser.

The solid antiperspirant product obtained using the method described above may have waxy solid appearance, and have a melting point in the range of 70-75° C. A penetration test using a Penetrometer (model no. 73515, from Precision Scientific Petroleum Instruments), loaded with 185 grams, was performed on samples of the solid antiperspirant product obtained using the method described above, with results showing a 5-10 mm penetration.

It has been observed by the inventors that in certain embodiments, good results may be obtained when part of the humectant is mixed initially with the antiperspirant drug active, and then part is mixed with the emulsifier ingredients and added after the pH adjuster has been mixed into the batch.

It has also been observed that in certain embodiments, a better product may be obtained with the use of multiple emulsifiers, for example, a fatty acid such as sodium stearate, and an alkoxylated alcohol such as steareth-20, instead of using either one alone. Embodiments such as that of Table 1 also include the fatty acid component present in a quantity that is at least two times that of the alkoxylated alcohol component.

A variety of modifications may be made to the process described above. For example, in certain embodiments, the order of adding certain ingredients may be modified. Also, in certain embodiments, some of the temperatures of the various processing operations may be modified. Modification in the quantities of various ingredients, for example, the drug active or the neutralizer, may vary properties such as the pH. Certain preferred embodiments have a pH in the range of about 8-9.5.

It should be noted that the classification of ingredients in compositions such as cosmetics may be somewhat arbitrary. For example, propylene glycol may be classified as a humectant, a solvent, and as a viscosity modifier. Consequently, for purposes of the descriptions herein, the components are characterized according to what is believed to be a useful role in providing the desired properties as used in compositions as described above.

One or more of the following advantages may also be present in certain embodiments of the present invention, including: (1) smooth application to skin; (2) a minimum of clumping during application; (3) minimal resistance during application; (4) translucent appearance; (5) a minimum of white residue formation on skin and clothes; (6) use of water leads to advantages in ease of manufacturing and cost savings.

It will, of course, be understood that modifications of the present invention, in its various aspects, will be apparent to those skilled in the art. The scope of the invention should not be limited by the particular embodiments described above. Other embodiments are also possible, their specific features depending upon the particular application. For example, embodiments of the present invention may be applicable to a variety of other types of cosmetic products for application to various parts of the body.

What is claimed is:

1. An antiperspirant composition in solid form, comprising:
an antiperspirant drug active;
water,
a humectant;
a fatty acid;
an alkoxylated alcohol; and
a neutralizer.

2. The composition of claim 1, wherein the neutralizer comprises an inorganic base.

3. The composition of claim 1, wherein the solid form is translucent.

4. The composition of claim 1, wherein the humectant comprises a glycol.

5. The composition of claim 1, wherein the fatty acid comprises sodium stearate and the alkoxylated alcohol comprises steareth-20.

6. The composition of claim 1, further comprising a fragrance.

7. The composition of claim 1, wherein the formulation has a pH in the range of 8 to 9.5.

8. The composition of claim 1 comprising:
1-25 weight percent of the antiperspirant drug active;
10-40 weight percent of the water,
25-60 weight percent of the humectant;
5-20 weight percent of the fatty acid;
2-10 weight percent of the alkoxylated alcohol; and
0.1-10 weight percent of the neutralizer.

9. The composition of claim 8, further comprising:
0.1-5 weight percent of fragrance.

10. The composition of claim 8, further comprising the fatty acid is present in an amount that is at least two times that of the alkoxylated alcohol.

11. The composition of claim 8, wherein the solid form is translucent.

12. The composition of claim 1 comprising:
1-25 weight percent of the antiperspirant drug active;
2-60 weight percent of the water,
25-60 weight percent of the humectant;
5-20 weight percent of the fatty acid;
2-10 weight percent of the alkoxylated alcohol; and
0.1-10 weight percent of the neutralizer.

13. The composition of claim 1, comprising:
6.500 weight percent of hydrous aluminum chlorohydrate as the antiperspirant drug active;
20.875 weight percent of the water,
52.750 weight percent of propylene glycol as the humectant;
12.000 weight percent of sodium stearate as the fatty acid;
5.000 weight percent of steareth-20 as the alkoxylated alcohol;
0.875 weight percent of sodium hydroxide as the neutralizer; and
2.000 weight percent of fragrance.

14. The composition of claim 13, wherein the solid form is translucent.

15. A method of forming an antiperspirant composition, comprising:
mixing in a first container a metal salt with water and a humectant to form a first liquid including dissolved metal salt therein;
adding a neutralizer comprising a base to the first liquid;
mixing in a second container a humectant, a fatty acid, and an alkoxylated alcohol to form a second liquid;
combining the second liquid and the first liquid including the neutralizer; and
solidifying the composition after the combining the second liquid and the first liquid.

16. The method of claim 15, further comprising adding a fragrance prior to the solidifying the composition.

17. The method of claim 15, further comprising using a glycol as the humectant in the first container and in the second container.

18. The method of claim 15, further comprising using sodium stearate and the fatty acid and using steareth-20 as the alkoxylated alcohol in the second container.
